(19) United States
(12) Patent Application Publication
(10) Pub. No.: US 2025/0043356 A1
(43) Pub. Date: Feb. 6, 2025

(54) COMPOSITION AND METHOD FOR DIAGNOSING BREAST CANCER USING EXTRACELLULAR VESICLE-MIRNA

(71) Applicant: University-Industry Foundation(UIF), Yonsei University, Seoul (KR)

(72) Inventors: Hyo-Il JUNG, Seoul (KR); Seung Il KIM, Seoul (KR); Sun Young PARK, Seoul (KR); Kyung-A HYUN, Bucheon-si (KR); Jee Ye KIM, Seoul (KR); Min Woo KIM, Seoul (KR); Young KIM, Cheonan-si (KR)

(73) Assignee: University-Industry Foundation(UIF), Yonsei University, Seoul (KR)

(21) Appl. No.: 18/481,035

(22) Filed: Oct. 4, 2023

(30) Foreign Application Priority Data
Oct. 5, 2022 (KR) 10-2022-0126760
Apr. 25, 2023 (KR) 10-2023-0053685

(51) Int. Cl. C12Q 1/6886; C12Q 1/6804; C12Q 1/6851

(52) U.S. Cl. CPC C12Q 1/6886; C12Q 1/6804; C12Q 1/6851; C12Q 2600/158; C12Q 2600/178

(57) ABSTRACT

Disclosed are a composition and a method for diagnosing breast cancer using extracellular vesicle-miRNA. The composition for diagnosing the breast cancer according includes an agent that measures an expression level of at least one miRNA selected from a group consisting of miR-9, MiR-16, MiR-21, and MiR-429. Furthermore, the method for diagnosing breast cancer includes measuring an expression level of at least one miRNA selected from a group consisting of MiR-9, MiR-16, MiR-21, and MiR-429 from a sample, and diagnosing the breast cancer based on the measurement result.

### CROSS-REFERENCE TO RELATED APPLICATION

This application claims priority from Korean Patent Application No. 10-2022-0126760 filed on Oct. 5, 2022 and Korean Patent Application No. 10-2023-0053685 filed on Apr. 25, 2023 in the Korean Intellectual Property Office, and all the benefits accruing therefrom under 35 U.S.C. 119, the contents of which in its entirety are herein incorporated by reference.

### SEQUENCE LISTING

The instant application contains a Sequence Listing which has been submitted in XML format and is hereby incorporated by reference in its entirety. Said XML copy, created on Jan. 14, 2024, is named “NCIP.P0121US_SequenceListing. XML” and is 4,542 bytes in size.

### BACKGROUND

### Field

The present disclosure relates to a composition and a method for diagnosing a breast cancer using extracellular vesicle-miRNA.

### Description of Related Art

MicroRNA (miRNA) is a small non-coding RNA that can control expression of a target mRNA and is involved in various physiological and developmental processes in cells. A miRNA expression level is frequently altered in cancer, thereby contributing to tumor growth, invasion, angiogenesis, and immune evasion. A circulating miRNA exists in plasma in various forms, including free miRNAs, lipoprotein-miRNA complexes, and miRNAs contained in extracellular vesicles (EVs). EV as a nanometer-sized lipid bilayer vesicle released from all cells is well known to not only play a key role in cell-cell communication but also transport various biomarkers such as proteins, mRNA, and miRNA. Because the lipid membrane of the EV protects the miRNA from ribonuclease degradation, the half-life in plasma of the miRNA contained in EV is longer compared to those of the other two types of miRNAs. Furthermore, a tumor-derived EV (TDE) and a cargo thereof have high specificity to cancer cells because they reflect the characteristics of origin thereof. Therefore, the EV containing the miRNA has been proposed as an ideal source for cancer diagnosis based on analysis of miRNA expression patterns (Patent Document 1).

Because the EV containing the miRNA is surrounded with the miRNA enriched in plasma, an essential step to analyze EV-derived miRNA for cancer diagnosis via liquid biopsy is to purify and concentrate cancer-related EV from the plasma. Ultracentrifugation (UC) as the most widely used EV isolation scheme is time-consuming, has low isolation efficiency, and has low selectivity from the cancer-related EV. A EV purification scheme based on capture due to affinity to surface epitopes of cancer cells has been used to overcome the limitation of the selectivity, but require complex processes including binding, washing, and concentrating.

Prior art literature to the present disclosure is Patent Literature: (Patent Document 1) Korea Patent Application Publication No. 10-2022-0025797 A (Mar. 3, 2022)

### SUMMARY

The present disclosure has been designed to solve the above problems. A purpose of the present disclosure is to provide a composition and a method for diagnosing a breast cancer which may isolate TDE using microfluidics which provides a continuous flow and engineering environment for molecular reaction to provide high throughput, high efficiency, and high selectivity, and may measure the miRNA from the isolated TDE, and may diagnose the breast cancer based on the measurement result.

A composition for diagnosing breast cancer according to the present disclosure comprises an agent that measures the expression level of at least one miRNA selected from the group consisting of miR-9, miR-16, miR-21, and miR-429.

A method for diagnosing breast cancer according to the present disclosure comprises measuring the expression of at least one miRNA selected from the group consisting of miR-9, miR-16, miR-21, and miR-429 from a sample and diagnosing the breast cancer based on the measurement result.

The composition for diagnosing breast cancer according to the present disclosure may serve as a biomarker based on a combination of the four types of miRNAs. According to the method for diagnosing breast cancer according to the present disclosure, early diagnosis of the breast cancer may be realized using liquid biopsy.

### BRIEF DESCRIPTION OF DRAWINGS

FIG. 1A is a schematic diagram of EpCAM and CD49f expressing extracellular vesicles (EVs) isolated from a plasma sample prepared using a horseshoe shaped orifice micromixer (HOMM) microfluidic chip.

FIG. 1B shows a profiling result of miRNA extracted from enriched EV using real-time PCR (identification of miRNA dysregulation in paired breast cancer and adjacent healthy tissue from TCGA database).

FIG. 1C shows an evaluation result of diagnosis possibility of a multi-miRNA panel of EV derived from a plasma of a breast cancer patient.

FIG. 2A is an image of the HOMM microfluidic chip.

FIG. 2B shows a scanning electron microscopy (SEM) image of MDA-MB-231 tumor-derived EV (TDE) after hydrodynamic isolation using a microfluidic chip (an enlarged SEM image shows that the TDE binds a surface of a 7-μm bead coated with EpCAM and CD49f-specific antibodies).

FIG. 2C shows a time-dependent TDE isolation efficiency using a microfluidic chip compared to a batch scheme of IP (immunoprecipitation).

FIG. 2D shows a flow cytometry result of EpCAM and CD49f-specific TDE after immunostaining with CD63 fluorescently labeled antibody.

FIG. 2E is a heat map of miRNA expression related to a breast cancer cell line.

FIG. 2F is a heatmap of miRNA expression related to specific EV enhanced by the microfluidic chip.

FIG. 3A shows a comparison result between Ct values of candidate endogenous genes of enriched EpCAM+ and CD49f+ EVs from a plasma sample.

FIG. 3B shows an analysis result of gene stability of miRNA.

FIGS. 4A to 4D show expression results of miRNA based on breast cancer subtype.

FIG. 4A: luminal A (estrogen receptor [ER]+, progesterone receptor [PR]+ and human epidermal growth receptor [HER-2]−).

FIG. 4B: luminal B (ER+, PR+, and HER-2+).

FIG. 4C: HER-2 (ER−, PR−, and HER-2+).

FIG. 4D: Triple-negative breast cancer (ER−, PR−, and HER-2−)].

FIG. 5A shows ROC curve analysis results of 7 candidate miRNAs used to distinguish between a healthy control and a breast cancer patient.

FIG. 5A to FIG. 5G: ROC AUC (area under curve) values related to diagnostic biomarkers.

FIG. 5I: ROC curve analysis based on breast cancer subtypes (luminal A, luminal B, HER-2, and triple negative) using combinations of top four significant miRNAs

FIG. 6A shows miRNA-mRNA interaction network analysis related to a target gene (A) and FIG. 6B shows enriched gene ontology (GO) analysis (B).

FIG. 7A to FIG. 7F show NTA (Nanoparticle tracking analysis) quantification results of extracellular vesicles (EVs) extracted from a medium of a breast cancer cell line.

FIG. 7A: NTA distribution of MCF-7 EV.

FIG. 7B: NTA distribution of BT-474 EV.

FIG. 7C: NTA distribution of SK-BR-3 EV.

FIG. 7D: NTA distribution of MDA-MB-231 EV.

FIG. 7E: EV amount measurement by NTA.

FIG. 7F: Western blot analysis of EpCAM, CD49f, CD9, CD81 and Alix in EV.

FIG. 8A and FIG. 8B show endogenous control evaluation results to compare microRNA (miRNA) expression levels of breast cancer cells (BT-474, MCF-7, SK-BR-3, and MDA-MB-231) and EVs with each other [heat-map of correlations between miRNA expression in cell lines and cell-derived EVs related to miR-16, miR-21, miR-9, miR-429, miR-96, miR-155, and miR-128 normalized by miR-484, miR-let7a, and miR-16 (the darker, the stronger the positive correlation (Pearson correlation coefficient is close to 1))].

FIG. 9A and FIG. 9B show evaluation results of hemolysis of a plasma sample before isolation of EVs to minimize hemolysis-induced miRNA expression inhibition [(FIG. 9A) a standard curve of hemolysis at absorbance 414 nm; (FIG. 9B) exclusion condition based on RBC concentration in 32 representative plasma samples of an entire population].

### DETAILED DESCRIPTION

Advantages and features of the present disclosure, and a method of achieving the advantages and features will become apparent with reference to embodiments described later in detail together with the accompanying drawings. However, the present disclosure is not limited to the embodiments as disclosed below, but may be implemented in various different forms. Thus, these embodiments are set forth only to make the present disclosure complete, and to completely inform the scope of the present disclosure to those of ordinary skill in the technical field to which the present disclosure belongs, and the present disclosure is only defined by the scope of the claims.

In interpreting a numerical value, the value is interpreted as including an error range unless there is no separate explicit description thereof.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein. In general, the nomenclature used in the present disclosure and the experimental methods described below are well known and commonly used in the art.

The term “diagnosis” used in the present disclosure means, in a broad sense, determining the actual condition of a patient's disease in all aspects. The name of the disease, etiology, type, severity, detailed condition of the disease, and the presence or absence of complications may be determined. The diagnosis in the present disclosure may include determining the onset or progression stage of the breast cancer, or differential diagnosis of subtypes of breast cancer.

The term “biomarker” used in the present disclosure refers to an indicator that may identify changes in the body, and may refer to a substance that may diagnose the normal or pathological state of the organism, the breast cancer, response to drugs, etc. in a distinguished manner from a normal control group. The biomarker may include organic biomolecules such as polypeptides or nucleic acids (e.g., mRNA, etc.), lipids, glycolipids, glycoproteins, and sugars (monosaccharides, disaccharides, oligosaccharides, etc.) that may increase or decrease in the breast cancer patient group compared to the normal control group.

The present disclosure discloses a composition for diagnosing breast cancer, the composition comprising an agent that measures the expression level of at least one miRNA selected from the group consisting of miR-9, miR-16, miR-21, and miR-429. The agent may include all kinds of genetic materials that may express the miRNA, bind complementarily to the miRNA, or amplify the miRNA, such as sense and antisense primers or probes that bind complementarily to the miRNA. In this regard, the miRNA may include at least one selected from the group consisting of miR-9 (SEQ ID NO: 1), miR-16 (SEQ ID NO: 2), miR-21 (SEQ ID NO: 3) and miR-429 (SEQ ID NO: 4) (see Table 1).

TABLE 1

| Clone | | SEQ ID |
|---|---|---|
| name | Nucleic Sequence | NO: |
| miR-9 | UCUUUGGUUAUCUAGCUGUAUGA | 1 |
| miR-16 | UAGCAGCACGUAAAUAUUGGCG | 2 |
| miR-21 | UAGCUUAUCAGACUGAUGUUGA | 3 |
| miR-429 | UAAUACUGUCUGGUAAAACCGU | 4 |

The extracellular vesicle may include a surface marker derived from the breast cancer. In this regard, the marker may be selected from the group consisting of CD49f, EpCAM, CD9, CD81, and Alix.

Furthermore, the composition for diagnosing breast cancer according to the present disclosure may be used to diagnose a subtype of breast cancer selected from the group consisting of luminal A, luminal B, HER-2, and triple-negative breast cancer.

The present disclosure discloses a breast cancer diagnostic kit, which may include the composition for diagnosing breast cancer. That is, the kit may include a tool, a reagent, etc. commonly used in RNA expression analysis that may measure the expression level of at least one miRNA selected from the group consisting of miR-9, miR-16, miR-21, and miR-429. For example, the ki may include a RNA extraction kit, a reverse transcription kit, a PCR (Polymerase Chain Reaction) reagent, a detection kit, etc. In the RNA extraction kit, the miRNA may be extracted from a patient's sample using a microfluidic chip according to the present disclosure, and other general methods may be used. The reverse transcription kit may reverse-transcribe the extracted RNA into complementary DNA (cDNA) and may analyze cDNA. In this case, means that may amplify a genetic substance, such as PCR or loop-mediated isothermal amplification may be used. When using the PCR, a substance including a primer and Taq polymerase is required. The detection kit may visualize and quantify the amplified DNA to check the expression state. Furthermore, the expression level may be checked in real time using real-time PCR.

The present disclosure discloses a method for diagnosing breast cancer, wherein the method may include following steps: (i) isolating extracellular vesicles from a sample using a microfluidic chip; (ii) isolating miRNA from the extracellular vesicle; (iii) measuring the expression of the miRNA; and (iv) determining the breast cancer from the expression result of the miRNA. In this regard, the miRNAs in the (iv) may be at least one selected from the group consisting of miR-9 (SEQ ID NO: 1), miR-16 (SEQ ID NO:2), miR-21 (SEQ ID NO:3), and miR-429 (SEQ ID NO:4) (see Table 1).

In the (i), the extracellular vesicles may be isolated via following steps: (i-1) injecting the sample and microbeads coated with antibodies targeting tumor-specific markers into the microfluidic chip; (i-2) isolating the microbeads from the sample that has passed through the microfluidic chip; and (i-3) isolating the extracellular vesicles from the microbeads.

### Example 1. Method

### 1-1. Cell and Clinical Sample Preparation

Four breast cancer cell lines (MCF-7, BT-474, SK-BR-3, and MDA-MB-231) were obtained from ATCC. These cell lines are distinguished from each other based on heterogeneous subtypes: MCF-7 is luminal A, BT-474 is luminal B, SK-BR-3 is HER-2, and MDA-MB-231 is TNBC (triple-negative breast cancer). The breast cancer cells were grown in RPMI medium containing 10% FBS at 70 to 80% confluency. The medium was removed therefrom, the cells were washed three times with PBS, and then grown in serum-free medium. After incubation of the cells at 37° C., under 5% CO2 and for 48 h, a conditioned medium was harvested, and was subjected to centrifugation once at 600 g for 30 min to remove the cells therefrom. EVs were further concentrated in cell-free supernatant using a 30K Macrosep Advance Centrifugal Device (Pall Life Science).

A total of 82 plasma samples were collected in accordance with the Yonsei University College of Medicine Independent Ethics Committee guidelines (IRB No. 4-2020-0350). An agreement that the blood samples are used only for research purposes was obtained from patients. To assess plasma sample quality, hemolysis was assessed prior to EV isolation. Details of 82 individuals, including 62 breast cancer patients and 20 healthy controls, are shown in a following Table 2:

TABLE 2

| | Breast cancer | Healthy |
|---|---|---|
| | patients (n = 62) | controls (n = 20) |
| Age | | |
| <50 | 31 (50.0) | 10 (50.0) |
| ≥50 | 31 (50.0) | 10 (50.0) |
| Stage | | |
| Early stage | 55 (88.7) | |
| Local metastasis | 7 (11.3) | |
| Tumor size | | |
| Smaller than 2 cm | 42 (67.7) | |
| Larger than or equal | 20 (32.3) | |
| to 2 cm | | |
| Lymph node metastasis | | |
| No | 51 (82.3) | |
| Yes | 11 (17.7) | |
| Subtype | | |
| Luminal A | 17 (31.8) | |
| Luminal B | 14 (18.2) | |
| HER-2 | 16 (27.3) | |
| Triple-Negative | 15 (22.7) | |

### 1-2. Design of Microfluidic Chip and Isolation of Extracellular Vesicles

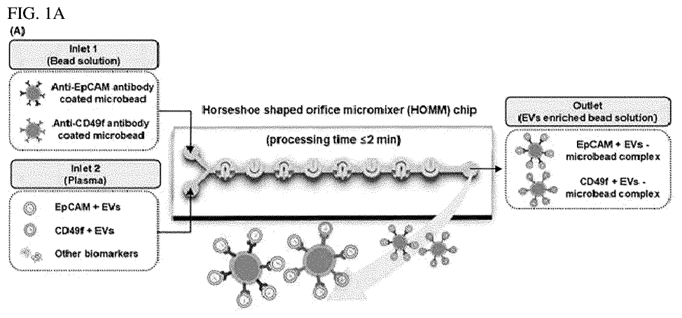

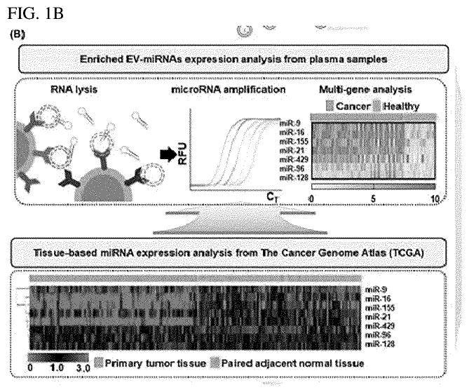

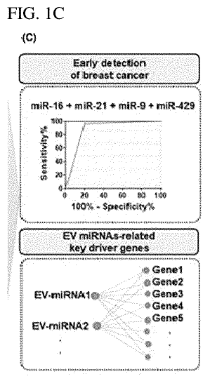

This microfluidic chip is composed of 150 continuous horseshoe-shaped channels to enhance collisions between EVs and microbeads coated with tumor-specific antibodies, thereby increasing the chance of binding interactions. Inside the microfluidic chip, tumor-specific EVs were captured from the culture medium or plasma, and the tumor-specific surface marker CD49f and the epithelial cell-specific marker EpCAM were isolated with within 2 minutes. After the microfluidic chip works, the microbeads carrying tumor-specific EVs were isolated in a centrifuged manner, and the supernatant was removed therefrom. Then, suspension buffer (Invitrogen, Pleasanton, CA, USA) was added to the concentrated microbeads in order to store the tumor-specific EVs while reducing RNA degradation (see FIG. 1B).

### 1-3. Characterization of Isolated Extracellular Vesicles

A specimen was fixed in Karnovsky's fixative (2% glutaraldehyde, and 2% paraformaldehyde in 0.1 M phosphate buffer, pH 7.4) for 24 hours and washed twice in 0.1 M PB for 30 minutes. The specimen was post-fixed in 1% OsO4 for 2 h and was dehydrated in a series of gradually concentration-increasing ethanol (50 to 100%) using a critical point dryer (CPD300, LEICA,). The specimen was coated with platinum using an ion sputtering device (ACE600, LEICA) and was observed with a field emission scanning electron microscope (SEM; MERLIN, Carl Zeiss).

A protein concentration was measured at 280 nm for comparison with EVs isolated from immunoprecipitation (IP) and the microfluidic chip. EVs binding to the microbeads in the medium of the breast cancer cells (MCF-7, SK-BR-3, BT-474, Hs578T, and MDA-MB-231) were measured via flow cytometry. The cells were washed in an ice-cold FACS buffer (PBS containing 1% BSA and 0.1% NaN3 sodium azide), and 5 L of fluorescent primary antibody anti-CD63-PE-Cy7 conjugated thereto were incubated for 30 min at 4° C. in the dark environment. To remove the non-specific binding, the cells were washed three times with FACS buffer, then and were measured using a FACS LSR II flow cytometer (BD, NJ, USA) and the measurements were analyzed with Flowing software v2.5.1.

### 1-4. Bioinformatic Analysis of Extracellular Vesicle-miRNA

Candidate diagnostic EV-miRNAs were profiled from miRNA expression of the tissues in TCGA as a public database that provides comprehensive cancer genomic profiles with important implications on biomarker discovery. To analyze the differential expression of miRNAs in the breast cancer, 85 candidate miRNAs were analyzed using 85 samples of cancer tissues and adjacent normal tissues of breast cancer patients registered in the TCGA database (see FIG. 1B). Adjacent normal tissue was collected under the judgment of an experienced doctor during the surgical removal of the breast cancer tissue from a breast cancer patient. Both cancer and adjacent tissue samples were collected before chemotherapy and treatment. A representation of the TCGA data related to the breast cancer samples used in this study is summarized in Table 3.

TABLE 3

| | Adjacent normal tissues | | | | Cancer tissues | | | | p-value |
|---|---|---|---|---|---|---|---|---|---|
| | Median (min-max) | | Mean (S.D.) | | Median (min-max) | | Mean (S.D.) | | (paired t-test) |
| hsa-miR-16 | 308 | (92.8-1709) | 435 | (331.9) | 511 | (145.9-3952) | 628.4 | (472.3) | 0.006 |
| hsa-miR-21 | 50117 | (10121-196399) | 54433 | (32501) | 248171 | (34749-467924) | 240079 | (87849) | <0.0001 |
| hsa-miR-9 | 250 | (49.1-2628) | 414.5 | (473) | 406.5 | (44.7-51986) | 3083 | (8477) | 0.0047 |
| hsa-miR-429 | 15.1 | (0-149.5) | 20.9 | (21.9) | 94.2 | (101-436.3) | 120.8 | (87.4) | <0.0001 |
| hsa-miR-96 | 3.2 | (0-36.2) | 4.5 | (4.5) | 26.8 | (2.4-153.9) | 41.7 | (34.8) | <0.0001 |
| hsa-miR-155 | 137 | (39.9-889.4) | 176 | (135.4) | 294 | (91.5-4114) | 484 | (589.4) | <0.0001 |
| hsa-miR-128 | 41.3 | (24.3-376.9) | 49.9 | (40.2) | 63.0 | (23.7-164.3) | 71.7 | (30.7) | <0.0001 |

We predicted candidate target RNAs of EV-miRNAs via integrated analysis of RNA-seq and miRNA-seq datasets for breast cancer in the TCGA database. Gene Ontology (GO) enrichment analysis of target genes of differentially expressed miRNAs was implemented using the DAVID (Database for Annotation, Visualization, and Integrated Discovery) tool. GO-based pathway and functional annotation analysis showed that “miRNAs in the cancer-related pathways” were generally enriched in the significantly upregulated cancer-specific EV-miRNAs in breast cancer tissues. GO terms were indicated with P<0.05 (see FIG. 1C).

1-5. Analysis of microRNA Expression and Multiple Panels of Extracellular Vesicles

EV-miRNA isolated from the microfluidic chip was extracted with the Total Exosome RNA and Protein Kit (Invitrogen) according to the manufacturer's protocol. The concentration of RNA was measured with a NanoDrop 3000 spectrophotometer (Thermo Fisher Scientific, Waltham, MA, USA). RT-qPCR was performed with TaqMan miRNA assay (Applied Biosystems). Briefly, reverse transcription was performed in the CFX96 Real-Time PCR System (Bio-Rad) using 5 ng of total miRNA, and 2 μL of cDNA in a final volume of 10 μL was used. Each point was evaluated in triplicate.

### 1-6. Statistical Analysis

To check effective reference genes for the expression of circulating miRNAs, we profiled EVs of miR-484, miR-let-7A, and miR-16 in breast cancer cell lines. Delta CT, GeNorm (https://genorm.cmgg.be), NormFinder (https://moma.dk/normfinder-software) and BestKeeper (https://www.gene-quantification.de/bestkeeper.html) software were used, and a P value <0.05 was considered statistically significant. The expression of miRNA from EVs in paired tumor and adjacent normal tissues in breast cancer patients and healthy control groups from TCGA was analyzed using two-tailed Student t-tests. The ROC (receiver operating characteristic) AUC (area under curve) of 0.65 or greater and a P value <0.05 for each miRNA was considered a candidate biomarker for breast cancer diagnosis. Logistic regression analysis was applied to build a model composed of candidate diagnostic miRNA groups.

### Example 2. Identification and Characterization of Extracellular Vesicles Derived from Breast Cancer Cells

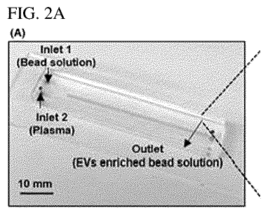

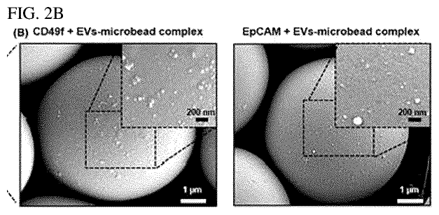

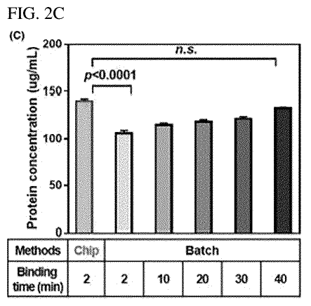

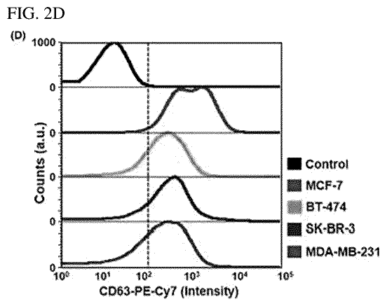

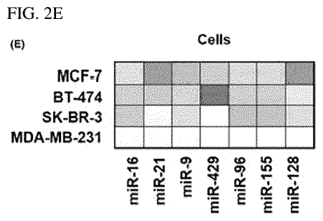

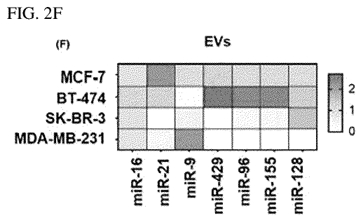

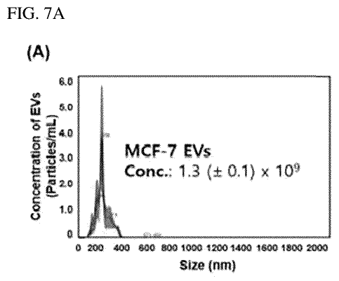

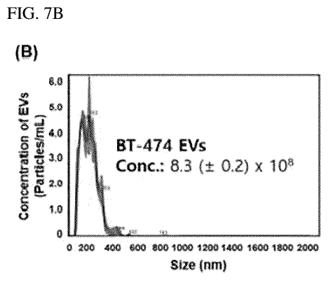

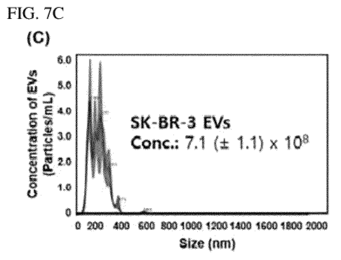

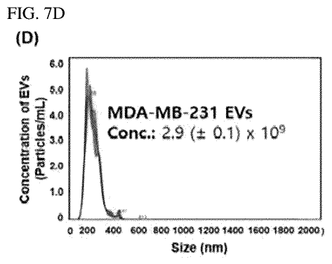

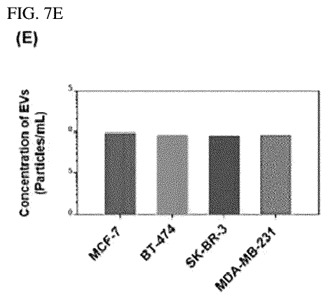

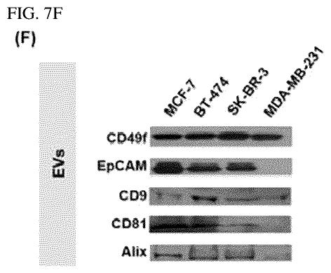

To characterize breast cancer-derived EVs, the total EV concentration of EVs from the cells was measured using a nanoparticle tracking analyzer, and the expression level of each of CD49f and EpCAM of the EV including the EV-specific markers (CD9, CD81, and Alix) was determined (see FIG. 7A to FIG. 7F). It was identified based on our previous report that general EV markers (Flotillin-1, CD63, and CD54) were expressed according to Western blot analysis of isolated EVs. Tumor-specific EVs were isolated using the present microfluidic chip, and the same concentration (109 particles/mL in PBS) was used (see FIG. 2A). The tumor-specific EVs isolated from the microbeads were identified in a SEM manner, and a size of the EV was in a range from 30 to 150 nm (see FIG. 2B). The microfluidic chip may isolate the breast cancer-specific EVs within 2 minutes. This is 20 times faster than the existing IP scheme (see FIG. 2C). The concentrations of the TDE isolated from the conditioned media of four types of breast cancer cell lines were 88.8% for MCF-7, 45.0% for BT-474, 53.4% for SK-BR-3, and 40.1% for MDA-MB-231 (see FIG. 2D).

### Example 3. Identification of miRNA Expression in Tumor-Specific Extracellular Vesicles

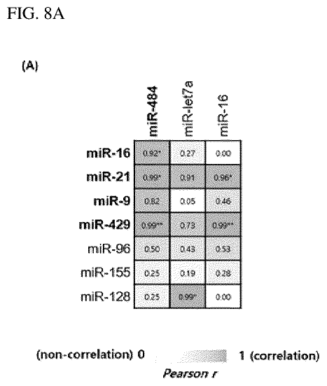

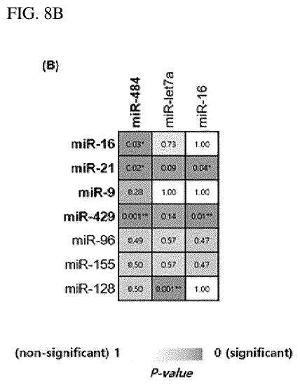

Candidate miRNAs for breast cancer were investigated using the TCGA database, in which 85 invasive breast carcinoma samples and a pair of normal tissue lesions have been registered (see Table 3). Seven types of miRNAs (miR-16, MiR-21, MiR-9, MIR-429, MIR-96, MiR-155, and MiR-128) known to be involved in cancer progression, including cell proliferation and angiogenesis were overexpressed in the cancer cells compared to the normal tissues. To check whether the nominated seven miRNAs related to the caner tissue were derived from the cells, the correlation between cancer cells and specific EVs was evaluated based on Pearson correlation. The miR-16, miR-21, and miR-429 were related to the most highly significant correlations between each cell and the EV (Pearson r >0.9 and P<0.05). It was assumed that the EV-miRNA was a major regulator affecting the cancer progression (see FIGS. 2E to 2F and FIGS. 8A to 8B). Additionally, miR-9, miR-96, miR-155, and miR-128 of the specific EVs may be overexpressed compared to those in those cells, thereby increasing the sensitivity of EV detection.

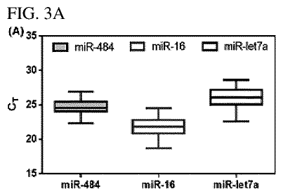

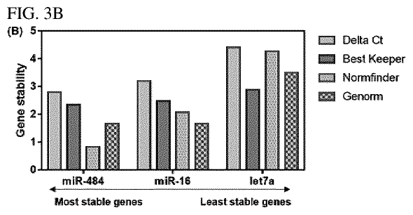

In order to identify potential liquid biopsy-based biomarkers for the breast cancer, the nominated target miRNAs (miR-16, miR-21, miR-9, miR-429, miR-96, miR-155, and miR-128) were validated using 82 plasma samples, including 62 breast cancer samples and 20 healthy controls. The miR-let7a and miR-16 are commonly used as endogenous controls for circulating biomarkers, and miR-484 was recently described as an EV endogenous control. We evaluated the expression of each miRNA normalized by these three candidates. The average Crs of miR-484, miR-16, and miR-let-7a from EVs were 24.7, 21.8, and 26.0, respectively, indicating that they are fairly reliable and enriched miRNAs as endogenous controls (see FIG. 3A). In the experimental setting, the miR-484 among these candidates was selected as the most stable endogenous control using four types of gene stability analysis tools (Delta Ct, Best keeper, Norm finder, and Genome) (FIG. 3B and Table 4).

TABLE 4

| | Ranking order (Better-Good-Average) | | |
|---|---|---|---|
| Methods | 1 | 2 | 3 |
| Delta Ct | miR-484 | miR-16 | let7a- |
| BestKeeper | miR-484 | miR-16 | let7a |
| Normfinder | miR-484 | — | let7a |
| Genorm | miR-484/miR-16 | miR-16 | let7a |

### Example 4. Characterization of miRNAs in Extracellular Vesicles Associated with Breast Cancer Subtype

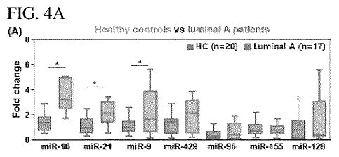

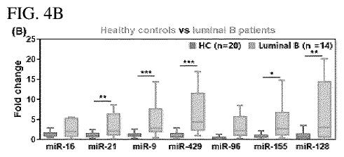

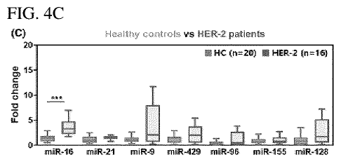

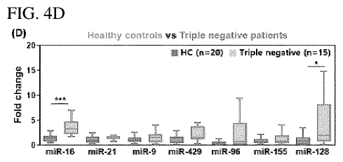

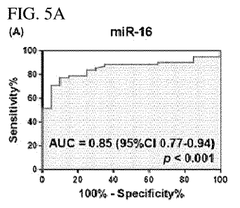

As expected, each miRNA in EV was associated with different tumor subtypes. We compared EV-miRNA expressions of healthy controls and patients with different subtypes of breast cancers with each other. Among the four statistically significant miRNAs (miR-16, miR-21, miR-9, and miR-429), miR-16 were closely related to luminal A, HER-2, and triple negative subtype. The miR-21 and miR-9 were closely related to luminal A and luminal B. The miR-429 was highly expressed in the luminal B subtype (FIGS. 4A to 4D).

### Example 5. Diagnostic Potential of Extracellular Vesicle miRNA in Breast Cancer

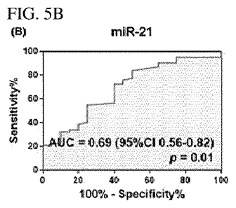

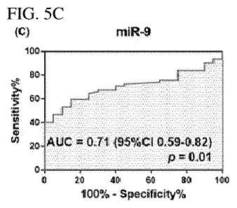

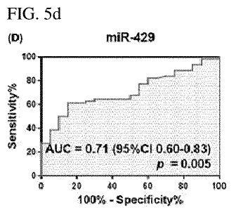

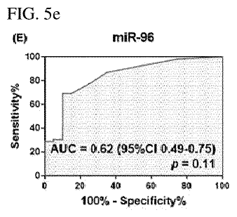

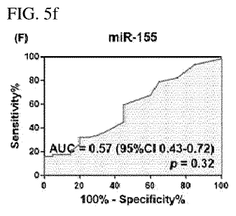

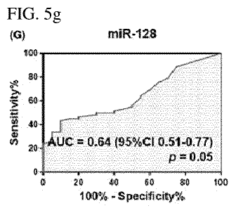

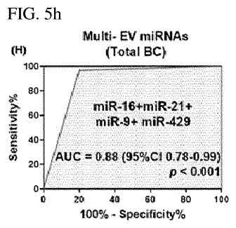

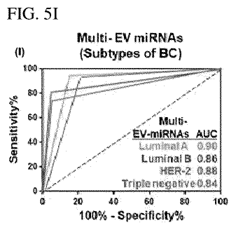

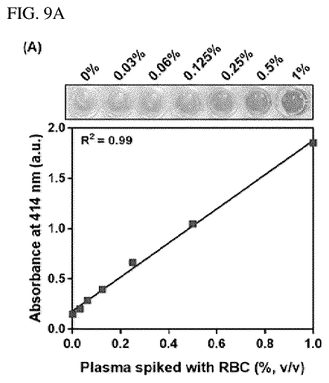

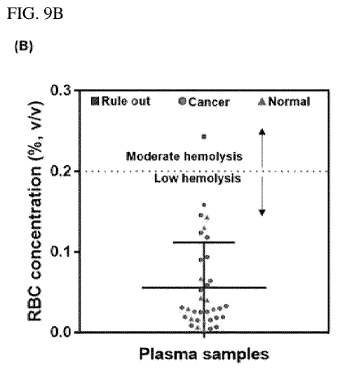

To investigate whether candidate miRNAs in microfluidic chip-enhanced EVs could serve as potential diagnostic biomarkers, miRNA expression was validated in 62 breast cancer patients and 20 healthy controls via quantitative RT-PCR (qRT-PCR; see FIG. 5A to FIG. 5I). Before using the plasma samples, it was identified that none of the samples showed hemolysis, which is known to significantly affect the miRNA measurements (see FIG. 9A and FIG. 9B). Next, the ROC curve was calculated to evaluate the diagnostic value of each miRNA, wherein the miRNA having a value >0.65 was interpreted as a good diagnostic biomarker. Among the seven candidates, four miRNAs (miR-16, miR-21, miR-9, and miR-429) were considered potential diagnostic biomarkers for the breast cancer (see FIG. 5A to FIG. 5G). The miR-16 possessed the highest diagnostic power for discriminating between breast cancer patients and healthy controls, with an AUC of 0.85 (95% confidence interval [CI], 0.77-0.94), followed by miR-21 (AUC, 0.70; 95% CI, 0.56-0.82), miR-9 (AUC, 0.71; 95% CI, 0.59-0.82), and miR-429 (AUC, 0.71; 95% CI, 0.60-0.83). The combination of these four miRNAs had a high sensitivity (96.8%) and a specificity (80%) with an AUC of 0.88 (95% CI, 0.78-0.99) (See FIG. 5H). The combination of the significant four miRNAs (miR-16, miR-21, miR-9, and miR-429) enabled discrimination between each subtype of breast cancer patients and healthy controls with an AUC of 0.90 (95% confidence interval [CI], 0.78-1.00) in the luminal A subtype, followed by luminal B (AUC, 0.86; 95% CI, 0.73-1.00), HER-2 (AUC, 0.88; 95% CI, 0.75-1.00), and triple-negative subtype (AUC, 0.84; 95% CI, 0.69-0.99) (See FIG. 5I).

Likewise, a previous study reports a significant correlation between miRNA profiles and intrinsic subtypes of breast cancer tumors. Thus, as each cargo in EV regulates the cancer microenvironment, the most significant miRNA in breast cancer-specific EV have the potential to predict cancer progression.

### Example 6. Functional Analysis for the Prediction of mRNA Targeted by Top Four miRNAs

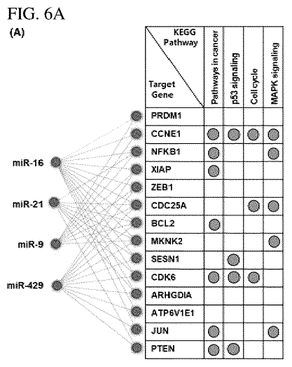

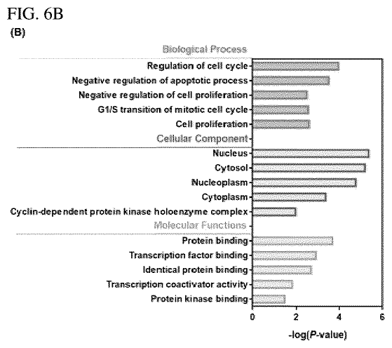

The mRNA targets that showed significant characteristics in EV-miRNA were portrayed by the miRNA-gene interaction analysis. These four miRNAs showed that the significant categories of biological procedures were “pathways in cancer”, “apoptosis,” and “cell cycle” (See FIG. 6A). The significant mRNA targets were then analyzed by GO analysis. GO biological processes include regulation of the cell cycle (GO: 0051726), negative regulation of the apoptotic process and cell proliferation (GO: 0043066 and GO: 0008285), and G1/S transition of the mitotic cell cycle (GO: 0000082). Moreover, the molecular functions of the targeted mRNA were applied to protein binding (GO: 0005515), transcription factor binding (GO: 0008134), identical protein binding (GO: 0042802), transcription coactivator activity (GO: 0003713), and protein kinase binding (GO: 0019901).

To date, carcinoembryonic antigen (CEA) and cancer antigen 15-3 (CA 15-3) have been employed as tumor markers playing important roles in predicting therapy responsiveness. The limitation of CEA and CA15-3 is that their level in serum is rarely elevated for patients with early-stage breast cancer, but some oncologists still use them as tumor markers for predicting breast cancer. When we profiled miRNA expression in EV from breast cancer patients and healthy controls, we found that a combination of the top four miRNAs (miR-9, miR-16, miR-21, and miR-429) was optimal for predicting the breast cancer. The value of these four miRNAs as a potential breast cancer biomarker was identified based on the ROC curve analysis, and the sensitivity thereof was 96.8%, and the specificity thereof was 80.0%, which were superior to those of conventional diagnostic schemes.

Some researchers also suggested that the combination of miRNAs in the blood can be useful to detect early-stage breast cancer by examining the dysregulated miRNA expression patterns in serum or plasma. As revealed in most studies, the diagnostic value of miRNA in liquid biopsy has become very clear, but the investigations of the specific EV-miRNA for early-stage breast cancer have been limited due to the lack of a reliable isolation method of the EV. The present inventors believe that the present analytical tool in association with the precise isolation of TDE has the potential to overcome some diagnostic limitations, such as sensitivity and selectivity in the field of EV-based in vitro diagnostics.

Although the embodiments of the present disclosure have been described in more detail with reference to the accompanying drawings, the present disclosure is not necessarily limited to these embodiments, and may be modified in a various manner within the scope of the technical spirit of the present disclosure. Accordingly, the embodiments as disclosed in the present disclosure are intended to describe rather than limit the technical idea of the present disclosure, and the scope of the technical idea of the present disclosure is not limited by these embodiments. Therefore, it should be understood that the embodiments described above are not restrictive but illustrative in all respects.

What is claimed is:

1. A composition for diagnosing breast cancer, the composition comprising an agent for measuring an expression level of at least one miRNA (micro RNA) selected from a group consisting of miR-9, miR-16, miR-21, and miR-429.

2. The composition of claim 1, wherein the miRNA is derived from an extracellular vesicle (EV).

3. The composition of claim 2, wherein the extracellular vesicle includes a breast cancer-derived surface marker.

4. The composition of claim 3, wherein the marker includes one selected from a group consisting of CD49f, EpCAM, CD9, CD81, and Alix.

5. The composition of claim 1, wherein the composition is used to diagnose a breast cancer of a subtype selected from a group consisting of luminal A, luminal B, HER-2, and triple-negative breast cancer.

6. The composition of claim 1, wherein the miR-9 is composed of a nucleic sequence of SEQ ID NO: 1.

7. The composition of claim 1, wherein the miR-16 is composed of a nucleic sequence of SEQ ID NO: 2.

8. The composition of claim 1, wherein the miR-21 is composed of a nucleic sequence of SEQ ID NO: 3.

9. The composition of claim 1, wherein the miR-429 is composed of a nucleic sequence of SEQ ID NO: 4.

10. A kit for diagnosing breast cancer, the kit comprising the composition according to claim 1.

11. A method for diagnosing breast cancer, the method comprising measuring an expression level of at least one miRNA (micro RNA) selected from a group consisting of MiR-9, MiR-16, MiR-21, and MiR-429 from a sample, and diagnosing the breast cancer based on the measurement result.

12. The method of claim 11, wherein the miR-9 is composed of a nucleic sequence of SEQ ID NO: 1.

13. The method of claim 11, wherein the miR-16 is composed of a nucleic sequence of SEQ ID NO: 2.

14. The method of claim 11, wherein the miR-21 is composed of a nucleic sequence of SEQ ID NO: 3.

15. The method of claim 11, wherein the miR-429 is composed of a nucleic sequence of SEQ ID NO: 4.

16. The method of claim 11, wherein the sample contains extracellular vesicles.

17. The method of claim 16, wherein measuring the expression level of the at least one miRNA (micro RNA) selected from a group consisting of MiR-9, MiR-16, MiR-21, and MiR-429 from a sample, and diagnosing the breast cancer based on the measurement result include:
isolating the extracellular vesicles from the sample using a microfluidic chip;
isolating the miRNA from the extracellular vesicles;
measuring the expression level of the miRNA; and
determining the breast cancer based on the measured expression level of the miRNA,
wherein the miRNA includes at least one selected from the group consisting of the miR-9, miR-16, miR-21, and miR-429.

18. The method of claim 17, wherein isolating the extracellular vesicles from the sample includes:
injecting the sample and a microbead coated with an antibody targeting a tumor-specific marker into the microfluidic chip;
isolating the microbead from the sample having passed through the microfluidic chip; and
isolating the extracellular vesicles from the microbead.


SEQUENCE LISTING (4 sequences)

SEQ ID NO: 1 - RNA, 23 nt, unidentified
  source 1..23: mol_type = other RNA
tctttggttatctagctgtatga

SEQ ID NO: 2 - RNA, 22 nt, unidentified
  source 1..22: mol_type = other RNA
tagcagcacgtaaatattggcg

SEQ ID NO: 3 - RNA, 22 nt, unidentified
  source 1..22: mol_type = other RNA
tagcttatcagactgatgttga

SEQ ID NO: 4 - RNA, 22 nt, unidentified
  source 1..22: mol_type = other RNA
taatactgtctggtaaaaccgt